(19) United States
(12) Patent Application Publication
(10) Pub. No.: US 2024/0366129 A1
(43) Pub. Date: Nov. 7, 2024

(54) SAMPLE HOLDER WITH CONDUIT FOR RECEIVING A SAMPLE

(71) Applicant: Covaris, LLC, Woburn, MA (US)

(72) Inventors: Amos G. Cruz, Wrentham, MA (US); Minh Duong, Gardner, MA (US); Jonathan O'Keefe, North Attleboro, MA (US); Annemarie Watson, Westwood, MA (US); Edward Ognibene, Belmont, MA (US); Ulrich Thomann, Stow, MA (US); Eugenio Daviso, Wrentham, MA (US)

(73) Assignee: Covaris, LLC, Woburn, MA (US)

(21) Appl. No.: 18/637,702

(22) Filed: Apr. 17, 2024

Related U.S. Application Data
(60) Provisional application No. 63/499,280, filed on May 1, 2023.

(51) Int. Cl. A61B 5/15

(52) U.S. Cl. CPC A61B 5/150351

(57) ABSTRACT

A sample holder for collecting a liquid sample, such as blood. The holder can include a vessel, a conduit having a proximal end extending from the vessel and a distal end positioned in the vessel, and a cap configured to engage with the vessel and create pressure to move a liquid sample in the conduit to move from the proximal end to the distal end. A desiccant can be used to dry a blood sample during storage.

### RELATED APPLICATIONS

This Application is a Non-Provisional application claiming priority under 35 U.S.C. § 119(e) to U.S. Application Ser. No. 63/499,280, filed May 1, 2023. The entire contents of this application are incorporated herein by reference in its entirety.

### BACKGROUND

### 1. Field of Invention

Methods and apparatus for sample holders.

### 2. Related Art

Sample holders, including individual tubes or vessels, are widely used in laboratory and other applications for collecting, holding and/or processing liquid samples, e.g., whole blood samples for diagnostic testing and research. Such vessels are used to expose a sample to a variety of different treatments, including exposure to sonic energy, heat/cooling cycles such as that used in PCR processing, and others.

### SUMMARY

In some embodiments, a sample holder includes a vessel having a wall defining an internal space. The vessel can be configured to receive and hold any suitable type of sample, such as a blood or other liquid sample. In some cases, the vessel can have an immobilizer such as a chemical material and/or physical structure suitable to interact with a blood or other liquid sample, e.g., to help keep the sample in a desired area of the vessel and/or treat the sample for storage or other processing.

In some embodiments, a sample holder includes a vessel having a wall defining an internal space and an opening to the internal space. For example, the vessel may be arranged as a tube with any suitable cross sectional shape, such as circular, rectangular, oval, etc., and may be made of any suitable material such as a polymer, metal, composite, etc. A conduit may have a distal end positioned in the internal space and a proximal end extending from the internal space, and may be configured to receive a liquid sample into the proximal end of the conduit. As an example, the conduit may include a capillary tube configured to draw or otherwise receive a blood or other liquid sample into the conduit by contacting the proximal end to a droplet or other collection of blood or other liquid material. A cap may be configured engage with the vessel such that movement of the cap relative to the vessel from a first position toward a second position causes a pressure differential between the proximal and distal ends of the conduit suitable to move a liquid sample from the conduit into the internal space. For example, movement of the cap relative to the vessel can create a suction or relatively low pressure in the internal space of the vessel to pull or otherwise move a liquid sample in the conduit toward the distal end so the sample is received into the vessel. In some cases, the cap may be configured to cause a pressure below ambient pressure in the internal space in response to movement of the cap relative to the vessel. In some embodiments, the pressure below ambient pressure may be suitable move a liquid sample from the conduit into the internal space via the distal end. Movement of the cap relative to the vessel may be in any suitable relative direction, e.g., the cap may be configured to cause the pressure differential in response to rotation and/or linear movement of the cap relative to the vessel.

To assist in generating a pressure differential, the cap may sealingly engage with one or more portions of the vessel. For example, the cap may be configured to sealingly engage with an outer surface of the wall of the vessel and/or an inner surface of the wall of the vessel. In some cases, the internal space of the vessel may be vented at least for some positions of the cap relative to the vessel, e.g., to permit a sample to be more easily moved from the conduit into the vessel. In some cases, a portion of the outer and/or inner surface of the wall of the vessel may include a channel that provides a vent for the internal space when the cap is at the first position, e.g., a position in which the cap and vessel are fully engaged with each other.

In some cases, the conduit may be fixed to the cap, e.g., so that the conduit moves with the cap when the cap is moved relative to the vessel. In some arrangements, the conduit may extend from an upper side of the cap and the cap may include a guard that extends from the upper side of the cap adjacent the proximal end of the conduit. Extension of the conduit from an upper side of the cap may make the proximal end of the conduit more accessible to a user, and the guard, e.g., one or more walls adjacent the proximal end, may help prevent accidental and/or unwanted contact with the conduit. In some embodiments, the cap and the conduit may be removable from the vessel, e.g., by moving the cap from the first position to the second position. As noted above, such movement may move a blood or other sample from the conduit into the internal space of the vessel, and thus a user may be permitted to move a sample from the conduit into the vessel and remove the cap and conduit from the vessel in one motion. In some cases, a cover can be configured to engage with the vessel to close the opening and seal the internal space closed after the cap and conduit are removed from the vessel. In some embodiments, a desiccant can be associated with the cover, and may be positionable in the internal space and configured to dry a blood sample enclosed in the internal space. As an example, the desiccant may extends from the cover and be configured to be positioned in the internal space with the cover engaged with the vessel.

In some cases, the sample holder may include a tray, stand or other holder configured to engage with the vessel and hold the vessel in an upright position on a surface with the proximal end of the conduit extending upwardly from the vessel. In some embodiments, the holder and the vessel may include complementary engagement features to removably engage the vessel with the holder. Such engagement features may help hold the vessel in place relative to the holder, such as in rotational directions and/or linear directions, e.g., where the vessel is resisted in movement about and/or along a longitudinal axis of the vessel. In some arrangements, the cap and the holder may be configured to support the vessel and conduit in a horizontal position on the surface with the conduit extending horizontally. Such an arrangement may permit the sample holder to be positioned horizontally so a user can provide a blood or other sample to the conduit in a horizontal direction.

In some embodiments, a method of collecting a blood sample includes providing a cap engaged with a vessel and conduit having a distal end within an internal space of the vessel. Blood or other liquid sample may be contacted with a proximal end of a conduit to draw the blood into the conduit, e.g., by capillary action, suction that pulls the sample into the conduit, pressure that pushes the sample into the conduit, etc. The cap may be removed from the vessel to create a pressure differential between the proximal end and the distal end of the conduit suitable to move the blood out of the conduit and into the internal space of the vessel. For example, movement of the cap relative to the vessel may create a relatively low pressure in the internal space of the vessel to cause a sample in the conduit to be moved into the vessel from the proximal end to the distal end. In some cases, removing the cap includes moving the cap relative to the vessel so that the cap engages with different portions of the vessel. For example, removing the cap may include engaging the cap with different portions of the vessel wall, e.g., the cap may engage with portions of the inner and/or outer surface of a wall of the vessel as the cap is moved relative to the vessel. In some cases, a seal on the cap may ride along or otherwise engage with a length of the vessel wall, e.g., an inner and/or outer surface of the wall, as the cap is moved relative to the vessel.

In some cases, contacting blood with the proximal end of the conduit includes venting gas from the internal space as blood or other liquid is drawn into the conduit. For example, venting gas can include venting gas through a space between the cap and the vessel wall, e.g., along a channel formed in the vessel wall and/or the cap. In some embodiments, removing the cap includes removing the conduit from the vessel with the cap, e.g., the conduit may be fixed to the cap and so be removed from the vessel when the cap is removed. In some arrangements, removing the cap includes rotating the cap relative to the vessel and/or moving the cap in a linear direction relative to the vessel.

In some embodiments, the method includes engaging a cover with the vessel to seal the internal space closed after the cap is removed from the vessel. Engaging the cover may include positioning a desiccant attached to the cover in the internal space, with the desiccant being suitable to dry the blood sample.

The sample holder may include any additional features described above or otherwise herein, and may do so in any arbitrary combination provided such features are not mutually exclusive. Other advantages and novel features of the invention will become apparent from the following detailed description of various non-limiting embodiments when considered in conjunction with the accompanying figures and claims.

### BRIEF DESCRIPTION OF THE DRAWINGS

Aspects of the invention are described with reference to the following drawings in which numerals reference like elements, and wherein:

FIG. 1 is perspective view of a sample holder in an illustrative embodiment;

FIG. 2 is a cross sectional view of the FIG. 1 sample holder with a cap engaged with a vessel of the sample holder;

FIG. 3 is a cross sectional view of the FIG. 1 sample holder with the cap disengaged from the vessel of the sample holder;

FIG. 4 is a perspective view of the vessel of the FIG. 1 sample holder with an associated cover;

FIG. 5 is a perspective view of the FIG. 1 sample holder including a holder for the vessel;

FIG. 6 is a front view of the FIG. 5 sample holder with the cap engaged with the vessel;

FIG. 7 is a perspective view of the FIG. 6 sample holder in a horizontal orientation on a surface;

FIG. 8 is a perspective view of a sample holder with cap configured to engage with an inner surface of the vessel in a horizontal orientation;

FIG. 9 is a cross sectional view of a sample holder in which the cap threadedly engages with a vessel holder;

FIG. 10 is a schematic view of the FIG. 9 sample holder with the cap engaged with the vessel holder;

FIG. 11 is a schematic view of the FIG. 9 sample holder with the cap removed from the vessel and a cover being engaged with the vessel;

FIG. 12 is a perspective view a holder for vessel and cap components of a sample holder;

FIG. 13 is a perspective view of the FIG. 12 embodiment with a vessel engaged with the holder in a vertical orientation;

FIG. 14 is a perspective view of the FIG. 13 embodiment with the cap disengaged from the vessel held by the holder in the vertical orientation; and

FIG. 15 is a perspective view of the FIG. 13 embodiment with a cover engaged with the vessel.

### DETAILED DESCRIPTION

It should be understood that aspects of the disclosure are described herein with reference to certain illustrative embodiments and the figures. The illustrative embodiments described herein are not necessarily intended to show all aspects of the disclosure, but rather are used to describe a few illustrative embodiments. Thus, aspects of the disclosure are not intended to be construed narrowly in view of the illustrative embodiments. In addition, aspects of the disclosure may be used alone or in any suitable combination with other aspects of the disclosure. For example, embodiments are described in which a desiccant is used with a cover to seal a vessel closed, and in which a holder is used to hold a vessel in one or more orientations. These features can be employed together in a sample holder, or can be used separately and without one or more of the others. For example, a cover need not include a desiccant, a sample holder need not include a holder (such as a tray or stand) for a vessel, and so on. This is just one example, and to the extent not mutually exclusive, other features described herein can be employed together or separately in various embodiments.

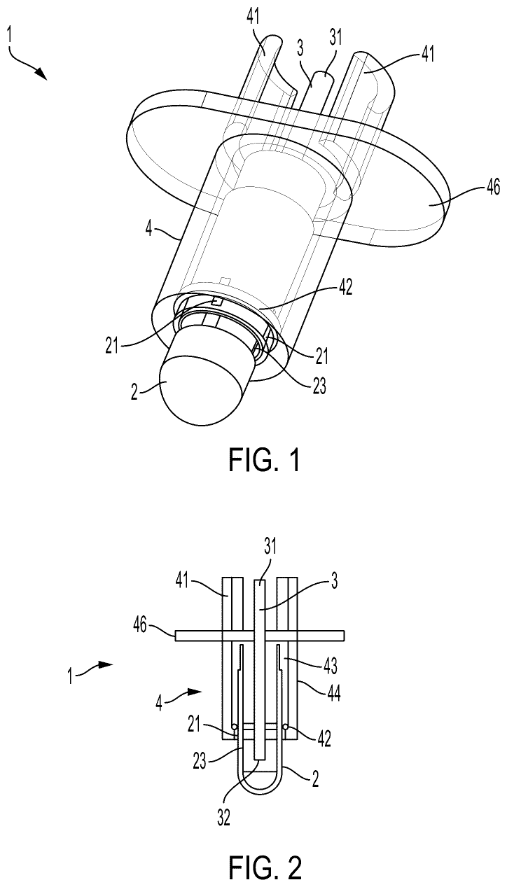

FIG. 1 shows an illustrative embodiment of a sample holder 1 that includes a vessel 2 and a cap 4 that can engage with the vessel 2, e.g., to close an internal space of the vessel 2 for receipt of a liquid sample such as blood into the vessel 2. In some embodiments, the cap 4 can be removably attached to the vessel 2, e.g., so the cap 4 can be removed from the vessel 2. In some cases, the sample holder 1 can be provided with the cap 4 engaged with the vessel 2, e.g., as shown in FIG. 1 so the sample holder 1 is ready to receive a sample. In some cases it may be preferable to separately package or otherwise provide the vessel 2 and cap 4, e.g., so a user assembles the vessel 2 and cap 4 together to an arrangement like that in FIG. 1 before use in collecting a sample. In some cases, the sample holder 1 includes a conduit 3 that is configured to receive a liquid sample into the conduit 3, e.g., by contacting the liquid material with a proximal end 31 of the conduit 3. In some embodiments, capillary action can draw the liquid material into the conduit 3, and the liquid material may flow into the conduit 3 so as to at least partially fill the conduit 3 from the proximal end 31 to a distal end 32 positioned in the internal space of the vessel 2. For example, the conduit 3 can include a capillary tube configured to draw a liquid sample, such as liquid blood, into the conduit 3 by contacting the liquid with the proximal end 31. In some cases, the conduit 3 can include a wick or other component that can receive a liquid sample (e.g., by wetting, capillary action, etc.). In some embodiments, a wick or other component can be contained within an outer tube or conduit. In some cases, the conduit 3 can include an anticoagulant or other material, e.g., to help prevent clotting or other disruption of flow of blood or other liquid in the conduit. The conduit 3 need not necessarily be configured to receive a sample by capillary action. Instead, the conduit 3 can receive a liquid sample by applying negative pressure to the distal end 32 of the conduit 3 (e.g., moving the cap 4 relative to the vessel 2 as discussed more below), or by applying positive pressure to urge the liquid sample to flow into the conduit 3 (e.g., by using a pipette to push a sample into the conduit 3). For example, the proximal end 31 can be contacted against a liquid sample and the cap 4 moved relative to the vessel 2 to move the sample into the conduit 3, e.g., by creating negative pressure at the distal end 32 of the conduit 3.

In some cases, the conduit 3 can be configured to collect a particular type of sample, such as liquid blood or other material, and the conduit 3 can be configured to collect a sample having a desired volume. For example, the conduit 3 can be configured to be completely filled with or filled to a particular extent with a liquid sample material and to have a particular internal volume so that the collected sample has a desired total volume. This arrangement can avoid any need for a user to measure sample material or otherwise take steps to collect a particular sample volume. In some cases, the conduit 3 can be visualized by a user, e.g., the vessel 2 and/or cap 4 can be made transparent, include a window or otherwise be configured so one or more portions of the conduit 3 can be visualized so the user can see the extent to which the conduit 3 is filled with sample. This can permit a user to confirm a volume of sample has been received by the conduit 3.

In some embodiments, a cap may be configured to permit a user to manipulate a vessel for collecting a sample and/or to support the vessel on a surface. For example, the cap 4 may have one or more portions that are be larger in size than the vessel 2, and so may provide a user with a larger or otherwise more effective gripping area than the vessel 2, e.g., so the user can more easily grasp and manipulate the cap 4 and vessel 2 to collect a blood sample. In some cases, the cap 4 may be configured to support the vessel 2 on a surface, such as a countertop, so that the vessel 2, and particularly the proximal end 31 of the conduit 3, are positioned above the surface on which the cap 4 is placed. In some cases, the cap 4 may include a grip 46 that can provide one or more gripping areas for a user to grasp and/or that can help support the vessel 2 in a desired way on a surface. As an example, the cap 4 and engaged vessel 2 may be placed on the surface in a horizontal orientation, e.g., such that the conduit 3 is oriented approximately horizontally or so that the conduit 3 is slightly inclined with the proximal end 31 above the distal end 32. A grip 46 may contact the surface to help support the cap 4 and vessel 2 on the surface. This may allow a user to prepare the sample holder 1 for sample collection and place the sample holder 1 on a surface while other actions are taken, such as piercing a subject's skin to produce a blood droplet for collection. Features on the cap 4 such as a size and/or shape of a grip 46 may help resist rolling or other movement of the sample holder 1 on the surface. For example, the grip 46 may have an oval shape as in FIG. 1 or otherwise configured so that the cap 4 and vessel 2 will not roll when placed on a surface, such as a table top. In some cases, the cap 4 may be configured to support the vessel 2 in other orientations, such as a vertical orientation in which the conduit 3 is oriented vertically with the proximal end 31 of the conduit 3 arranged above the distal end 32 of the conduit 3. For example, a grip 46 of the cap 4 may have a flat or otherwise configured surface to permit the cap 4 and vessel 2 to be placed on a surface so the conduit 3 is oriented vertically, e.g., the conduit 3 extends upwardly from the cap 4 and the vessel 2 extends below cap 4, or horizontally, e.g., so the conduit extends horizontally, without assistance from a user or other support. In some cases, a long edge of the grip 46 in FIG. 1 may contact a surface along with another portion of the cap 4 and/or a portion of the vessel 2 when the sample holder 1 is placed horizontally on a surface.

The conduit 3 can be supported or otherwise held in the vessel 2, e.g., so that a user can hold and manipulate the vessel 2 and/or cap 4 to contact the proximal end 31 of the conduit 3 to a sample material. This may help ease use of the sample holder 1 since the user need not handle a relatively small and delicate conduit 3. It may also help prevent contamination of a sample since a user may be less likely to touch or otherwise contact the conduit 3, e.g., at the proximal end 31. In some cases, the cap 4 may include guards 41, e.g., one or more wall sections that extend from an upper surface of the cap 4 and help prevent unwanted contact of the proximal end 31 with surfaces. In some cases, the guard(s) 41 can extend approximately a same extent as the conduit 3 from an upper surface of the cap 4 and may extend at least partially around or otherwise be positioned about a periphery of the conduit 3. For example, the guards 41 in FIG. 1 can have an arcuate shape when viewed from above and can each extend around the conduit 3 along an arcuate length of about 30 degrees to 90 degrees each (and may have a smaller or larger arcuate length). Thus, a portion of the conduit 3 can protrude from the cap 4, e.g., having a sufficient length to permit suitable contacting of the proximal end 31 with a liquid sample material. In some arrangements, the guards 42 may extend further from the cap 4 than the conduit 3, e.g., so the cap 4 and conduit 3 can be placed upside down with the guards 41 supporting the cap 4 and conduit 3 on a surface with the conduit oriented vertically with the proximal end 31 below the distal end 32 and so the proximal end 31 is out of contact with the surface. In some arrangements, the proximal end 31 of the conduit 3 may extend upwardly past the guards 41.

In some cases, the conduit 3 can be fixed to the cap 4, e.g., a portion of the conduit 3 can extend through an opening of the cap 4 that engages with the conduit 3 by friction fit, adhesive, one or more fasteners, etc. The conduit 3 can be supported so that the distal end 32 of the conduit 3 is positioned in a desired way in the vessel 2, e.g., so that the distal end 32 is positioned above a bottom of the vessel 2 when the cap 4 is fully engaged with the vessel 2, e.g., in the position shown in FIG. 1. This can help prevent the conduit 3 from drawing liquid material into the conduit 3 from the distal end 32, e.g., because the distal end 32 can be positioned above any liquid material in the vessel 2, such as reagents, stabilizers, etc. In some embodiments, the vessel 2 and cap 4 can be configured so that a user can see the conduit 3, e.g., while providing a sample into the conduit 3. For example, the conduit 3 and/or the vessel 2 and/or the cap 4 can include a transparent material or window that permits the user to see the conduit 3. This can help a user determine that the conduit 3 is full or otherwise has a suitable sample volume, e.g., preventing the user from overdrawing a sample.

To assist in allowing a sample to be moved into the conduit 3 from the proximal to the distal end when the cap 4 is engaged with the vessel 2, an internal space of the vessel 2 may be vented. That is, in some positions of the cap 4 relative to the vessel 2, the cap 4 may sealingly engage with the vessel 2, e.g., so that the internal space of the vessel 2 is completely closed except for a pathway through the conduit 3. As an example, the cap 4 may include a seal 42, e.g., located between an inner surface of a wall of the cap 4 and an outer surface of a wall of the vessel 2 so that an air-tight seal is created between the cap 4 and the vessel 2. However, one or more vents may be provided for at least some positions of the cap 4 relative to the vessel 2, such as with the vessel 2 fully engaged with the cap 4. Such a position is shown in FIG. 1, and the vessel 2 may include one or more channels 21 that provides a vent for the internal space of the vessel 2 when the cap 4 is at the position shown in FIG. 1. Thus, as fluid moves into the conduit 3 from the proximal end 31, displaced gas may exit the internal space of the vessel 2 via the channels 21. The channels 21 may be configured so that the seal 42 cannot form an airtight seal at least with a portion of the channel 21. Other vent configurations are possible for the vessel internal space, such as ports through the vessel wall, valves, etc. In some cases, the vessel may have a reduced diameter or other size in one or more portions, e.g., so that the seal 42 cannot contact or only loosely contacts the vessel 2 so an airtight seal cannot be created when the cap 4 is fully engaged with the vessel 2. For example, the seal 42 on the cap 4 may be positioned at a relatively small diameter portion of the vessel 2 when the cap 4 and vessel 2 are fully engaged. This may permit gas to flow in a space between the seal 42 and the vessel 2 to vent the internal space. However, upper portions of the vessel 2 may have a larger diameter to enable the seal 42 to create an airtight seal between the cap 4 and vessel 2.

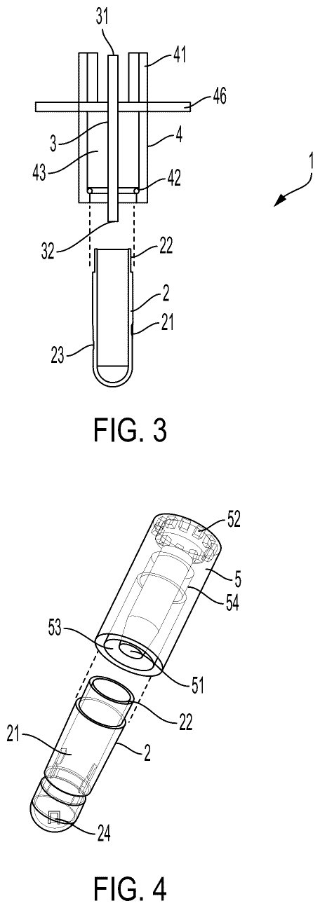

After a blood or other sample is drawn into or otherwise moved into the conduit 3, the cap 4 can be moved from a first position (e.g., where a seal 42 of the cap 4 is engaged where the channels 21 are located or a vent is otherwise provided for the internal space of the vessel) toward a second position (e.g., where the cap 4 is removed from the vessel 2 or at least partially moved from the fully engaged position). In some cases, the cap 4 can have a cavity 43 configured to receive at least a portion of the vessel 2, e.g., so that the distal end 32 of the conduit 3 is positioned in the vessel 2 as shown in FIG. 2. The cap 4 and vessel 2 can be configured so that when the cap 4 is moved from the first position (e.g., shown in FIG. 2) toward the second position, a pressure differential is created between the proximal end 31 and the distal end 32 of the conduit 3 to move a liquid sample in the conduit 3 into the vessel 2. For example, the seal 42 (such as an o-ring or other gasket) on the cap 4 can engage with the vessel 2 to create an airtight seal between the cap 4 and the vessel 2. As the vessel 2 is moved out of the cavity 43 of the cap 4, gas in the cavity 43 and the internal space of the vessel 2 (such as air) is put under a pressure below ambient because of the enlarged gas volume of the cavity 43 and the internal space of the vessel 2. The relatively low pressure in the cavity 43 and internal space can cause a sample in the conduit 3 to flow from the proximal end 31 to the distal end 32 of the conduit 3, thereby forcing a blood or other sample in the tube 3 to flow into the bottom of the vessel 2. Note that although channels 21 or other vent features are provided when the cap 4 is fully engaged with the vessel 2, as the vessel 2 is withdrawn from the cavity 43, the seal 42 can engage with the outer surface of the wall of the vessel 2 to form an airtight seal that causes fluid to flow into the proximal end 31 of the conduit 3. While in some embodiments the cap 4 includes a seal 42 to engage with the vessel 2, other arrangements are possible. For example, the cap 4 and vessel 2 can engage with a close or interference fit, the vessel 2 can have one or more seals that engage with an inner wall of the cavity 43 of the cap 4, and so on. In some cases discussed more below, a portion of the cap 4 may engage with an inner surface of the wall of the vessel 2, e.g., a seal between the inner surface of the vessel wall and an outer surface of a wall of the cap 4 may cause relatively low pressure to be created in the internal space of the vessel as the cap 4 and vessel 2 are moved relative to each other. In some cases, the vessel 2 and cap 4 can be configured to cause a volume of gas that is larger than the volume of the conduit 3 to flow through the conduit 3 as the cap 4 is moved from the first position (e.g., shown in FIG. 2) toward the second position (e.g., shown in FIG. 3) relative to the vessel 2. This can help ensure that a sample in the conduit 3 is completely discharged into the vessel 2. In some cases, the volume of gas moved through the conduit 3 can be two or more times the volume of the conduit, e.g., 3, 4, 5, 10 or more times the volume of the conduit 3. In cases where the conduit 3 includes a wick or other element that receives a sample by wetting, capillary action contained within an outer tube or conduit, relatively high volumes of gas moved through the conduit 3 by the cap 4 can assist in ensuring a suitable amount of sample is discharged from the conduit 3.

In some embodiments, after a blood or other sample is discharged from the conduit 3 into the internal space of the vessel 2, the opening of the vessel to the internal space can be sealed closed so that a blood or other sample can be contained in a sealed internal space of the vessel 2, e.g., so the blood or other sample is isolated from external environmental conditions such as humidity, oxygen, air, etc. For example, when the cap 4 is moved toward the second position relative to the vessel 2, e.g., as shown in FIG. 3, the cap 4 can be removed from the vessel 2. When the cap 4 is disengaged from the vessel 2, the internal space of the vessel 2 may be sealed closed by a cover 5 that covers the opening of the vessel 2, e.g., as shown in FIG. 4. In some cases, the cover 5 can be configured to receive an upper part of the vessel 2 into a cavity 53 of the cover 5. The cover 5 and vessel 2 may engage in any suitable way, such as by interference fit, adhesive, threaded engagement, and so on. In some cases, an upper portion of the vessel 2 may include threads 22 configured to engage with complementary threads 54 in the cavity 53 of the cover 5. In some cases, the portion of the vessel 2 including the threads 22 may have a reduced diameter or other size relative to the wall of the vessel 2 below the threads 22. This may help make removal of the cap 4 from the vessel 2 easier, e.g., because the seal 42 or other portions of the cap 4 may not engage with the threads 22. The cover 5 may include crenelations, grooves, tabs or other so-called automation features 52 that can help an automated system handle the cover 5 and vessel 2 and/or move the cover 5 relative to the vessel 2, e.g., for engagement and disengagement of the cover 5 with the vessel 2.

In some cases, the cover 5 can include a desiccant material 51, e.g., suitable for drying a blood or other liquid sample held in the vessel 2. A desiccant can include a molecular sieve such as a zeolite, a silica gel, or other suitable material for drying blood or other liquid samples. In some embodiments, a portion of the cover 5 can be made entirely of desiccant material. The desiccant 51 can be positioned in the vessel 2 so that the desiccant 51 is suitably close to a blood or other liquid sample held in the vessel 2, e.g., at the bottom of the vessel 2. In some cases, the desiccant 51 can be configured to fill the internal space of the vessel 2 to a desired extent, e.g., to take up 50%, 60%, 70%, 80% or more of the internal space of the vessel 2. This can help reduce the volume of the internal space, which may aid in drying of a blood sample. The sample holder 1 can include an immobilizer 24 such as a chemical material and/or structure in the internal space of the vessel 2 (e.g., at the bottom of the vessel 2) to immobilize or otherwise act on the blood or other sample, e.g., to help preserve or otherwise treat the sample for drying or later use. For example, the immobilizer 24 can increase a viscosity of a sample, enhance the sample's ability to adhere to the vessel 2 surface, capture portions of the sample in voids or other spaces (e.g., by surface tension, wetting or absorbance features), etc.

In some embodiments, a cover can be configured to lock onto a vessel, e.g., so the cover cannot be removed from the vessel or from a portion of the vessel. Such a configuration may help reduce contamination of a sample after the sample has been collected and/or confirm that a sample has not been exposed to an external environment since being collected. In some cases, a cover 5 can be configured to lock onto a vessel 2, either removably or non-removably. In some embodiments, a cover 5 may include one or more latches configured to engage with a portion of a vessel 2, e.g., such that once the latches are engaged with the vessel 2, the cover cannot be removed without disengaging the latches. In some cases, the latches may be configured as hooks that extend downwardly along one or more sides of the cover 5, e.g., downwardly from a bottom edge of the cover 5. The latches may be configured to resiliently bend inwardly, e.g., so that lower hook portions of the latches move radially inwardly toward the vessel 2. This may enable the hook portions to engage with the recess 23 on the vessel 2 or some other portion of the vessel 2. Thus, as the cover 5 is pressed downwardly onto the vessel 2, the lowermost surfaces of the hooks on the latches may contact the outer surface of the vessel wall until the lowermost part of the hooks are positioned at the recess 23. With the hooks positioned at the recess 23, the latches can bend inwardly, e.g., due to a resilient or elastic recovery, so the hooks engage with the recess 23. With the hooks engaged with the recess 23, the cover 5 may not be removed without disengaging the latches from the recess 23, e.g., by exerting suitable upward force on the cover 5 and/or bending the latches to disengage the hooks from the recess 23. In some cases, the cover 5 and vessel 2 may not be disengaged without using a suitable tool, e.g., to disengage the latches from corresponding vessel features, applying suitably large force and/or by cutting or otherwise damaging the latches. Thus, a tamper evident securement between the cover 5 and vessel 2 may be provided.

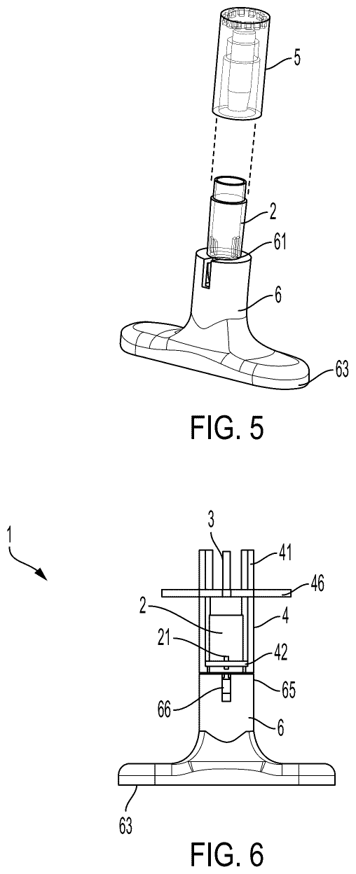

In some cases, a sample holder may include a holder, stand or other support for a vessel, e.g., that can support the vessel in two or more orientations. Such a stand may aid a user in manipulating, securing, or otherwise holding or using the sample holder during use. For example, FIG. 5 shows an embodiment in which a vessel 2 is received and held by a stand 6. The stand 6 can be configured in different ways to receive and hold a vessel 2, e.g., having an opening or cavity 61 to receive at least a portion of the vessel 2. For example, the lower portion of a vessel 2 can be received into the opening 61, e.g., so that the vessel 2 can be held in an upright orientation on a surface without aid from a user. In such a case, a base 63 of the stand 6 can be placed on the surface and can be configured to hold the stand 6 and vessel 2 in an upright orientation on the surface. In some cases, the stand 6 can engage the vessel 2, e.g., so that movement of the vessel 2 relative to the stand 6 in one or more directions is resisted. For example, in some cases the stand 6 may engage with engagement features on the vessel 2 to help resist rotation and/or longitudinal movement of the vessel 2 relative to the stand 6. FIG. 5 shows an illustrative embodiment in which the stand 6 includes features configured to engage with the lower portion of the vessel 2, e.g., to resist rotation and/or movement of the vessel out of the cavity 61 or otherwise relative to the stand 6. Engagement features on the stand 6 may take any suitable form and may be complementary to engagement features on the vessel 2. For example, in FIG. 5 the lower portion of the vessel includes a circumferential recess and ribs 23 (see FIG. 1) configured to engage with portions of the stand 6 to resist rotation of the vessel 2 in the cavity 61 and/or movement of the vessel 2 out of the cavity 61. For example, the cavity 61 may include an annular ring or ring sections configured to engage with the recess and ribs 23 to help keep the vessel 2 in the cavity 61 and/or resist rotation of the vessel 2. The stand 6 may resiliently engage with the vessel 2, e.g., a slot extending along a sidewall of the stand 6 near the cavity 61 may permit the wall portions that define the cavity 61 to flex outwardly to receive the vessel 2 and flex inwardly to engage the recess 23. However, the engagement features may be configured in other ways, e.g., recesses on the vessel 2 may be engaged by ribs or tabs on the stand 6, and so on. In some cases, the stand 6 may frictionally engage with the vessel 2, e.g., to resist rotational and/or longitudinal movement of the vessel 2 relative to the stand 6. For example, a portion of the stand 6 at the opening 61 or other area may frictionally engage the vessel 2 so the vessel 2 cannot be easily inserted into and/or removed from the opening 61. This may assist in ensuring the vessel 2 and stand 6 are not separated in an unwanted or unintentional way. As an example, and as shown in FIG. 5, this may permit a user to hold the stand 6 and place a cover 5 on the vessel 2.

Engagement of the stand 6 with the vessel 2 may aid in disassembly and/or assembly of the sample holder, collection of sample material, treatment of sample material in the vessel, etc. For example, in some cases, the stand 6 may engage the lower portion of the vessel to resist relative rotation, linear movement along a longitudinal axis of the vessel or other movement of the vessel 2 relative to the stand 6, e.g., by engaging the engagement features on the vessel and stand with each other. However, the upper portion of the vessel 2 and/or the cap 4 may not be directly engaged with the stand, e.g., such that the cap 4 can be moved relative to the lower portion of the vessel 2 and the stand 6. For example, the cap 4 may be fully engaged with the vessel 2 as shown in FIG. 6, and yet the vessel 2 may be engaged with the stand 6, e.g., at a recess 23. As a result, a user can move the cap 4 upwardly relative to the stand 6 and thereby cause a liquid sample in the conduit 3 to be moved into the internal space of the vessel 2 and remove the cap 4 from the vessel 2. This can expose the internal space of the vessel 2, e.g., permitting a user to access a sample in the vessel and/or to add material such as treatment reagents to the vessel. As noted above, the stand 6 may support the vessel 2 on a surface without assistance from a user, e.g., by resting the base 63 on the surface. In some cases, removal of the cap 4 from the vessel 2 may remove a conduit 3 from the vessel as well, e.g., as in FIG. 3. This can permit a cover 5 to be placed on the vessel 2, e.g., as in FIG. 5.

In some cases, the stand 6 may be configured to permit visualization or other observation of at least a portion of the vessel 2 when the vessel 2 is held by the stand 6. This may, for example, permit a user to confirm that a sample has been properly received into the vessel 2, e.g., after a cap 4 has been moved relative to the vessel 2 to move a sample from the conduit 3 into the vessel 2. For example, the stand 6 may be transparent and/or include a window 66 so a part of the vessel 2 can be observed by a user and/or optical detector. The window 66 may be formed as a cavity or opening in a portion of the stand 6, e.g., extending through a collar 65 that defines a part of the opening 61 into which the vessel 2 is received. In some cases, the collar 65 may have a tapered or concave portion around the opening 61, e.g., to help guide movement of a vessel 2 into the opening 61. In some cases, a lower portion of the vessel 2 may have a tapered, rounded or otherwise shaped portion to help guide the vessel 2 into the opening 61. The window 66 may provide a dual purpose, e.g., allowing visualization of the vessel 2 as well as allow portions of the collar 65 to move apart to receive the vessel 2 into the opening 61.

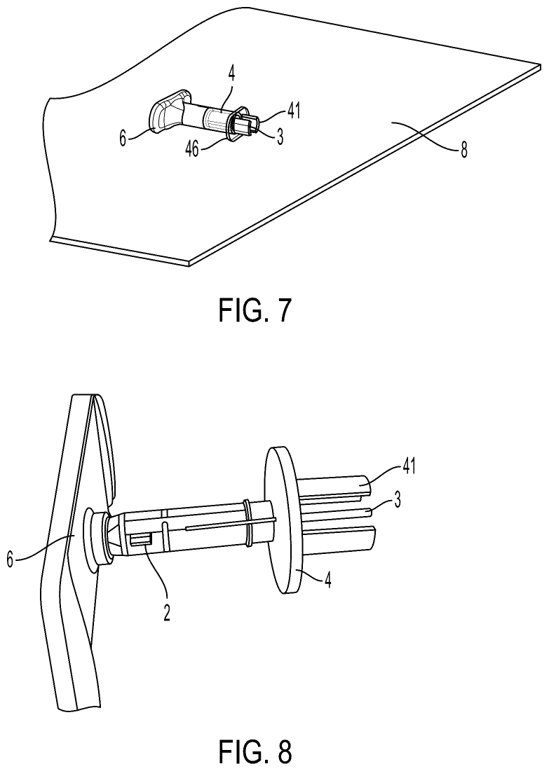

In some embodiments, the stand may be configured to aid a user in grasping and manipulating the stand 6 and an attached sample holder 1. For example, the stand 6 may be sized and shaped to be comfortably and accurately manipulated by hand by a user. Features of the stand 6 to assist in manipulation can vary as desired; in some cases the stand 6 may include finger grips, e.g., one or more concave portions below the collar 65 that are sized and shaped to permit a user's fingers to hold the stand 6. In some cases, the finger grips can be configured for gripping between a forefinger and thumb; in some cases, the finger grips can be configured to be positioned between adjacent fingers, e.g., so portions of an index and middle finger are engaged at the finger grips. This may permit a user to position the proximal end 31 of the conduit 3 at a sample source to provide a sample into the conduit 3, e.g., by capillary action. As noted above, the stand 6 may be configured to support the vessel 2 in multiple orientations, whether while the stand 6 is held by a user or the stand 6 is placed on a surface. One such orientation (e.g., a vertical orientation in which the conduit 3 is oriented vertically) is shown in FIG. 6 where the base 63 of the stand 6 may be placed on a surface such as a countertop to support the vessel 2 without user or other assistance. Another possible orientation (e.g., a horizontal orientation in which the conduit 3 is oriented horizontally) is shown in FIG. 7 where the stand 6 may be placed on its side on a surface 8 such as a countertop to support the vessel 2 without user or other assistance. In a horizontal orientation, a portion of the cap 4 such as a grip 46 may contact the surface 8 as well to support the holder 1 on the surface. Both orientations may be useful for different purposes, such as harvesting a sample, removing a cap from or placing a cap on a vessel, or others. As an example, with the vessel 2 and cap 4 in a horizontal orientation, a blood droplet on a user's finger may be placed adjacent the proximal end 31 of the conduit 3 so the blood sample can be received into the conduit 3. This may help a user steady the finger, e.g., by partially resting the finger on the surface 8, while positioning the blood droplet adjacent the proximal end 31.

FIG. 8 shows another sample holder 1 in a horizontal arrangement on a surface. FIG. 8 illustrates that embodiments can be configured to permit a user to visualize portions of the conduit 3 and/or vessel 2 interior, e.g., to confirm that a sample has been received by the conduit 3 and/or the vessel 2. In FIG. 8, the conduit 3 can be seen holding a dark liquid, e.g., the color and opacity of blood, and illustrates how a user can confirm that a sample has been received into the conduit 3. Although not shown in FIG. 8, if the cap 4 is removed from the vessel 2 so the liquid sample is received into the vessel 2, the user could visualize as well that the dark liquid is present in the vessel, e.g., because the vessel 2 is made of a transparent material, has a window or otherwise is configured to permit a user to see into the internal space.

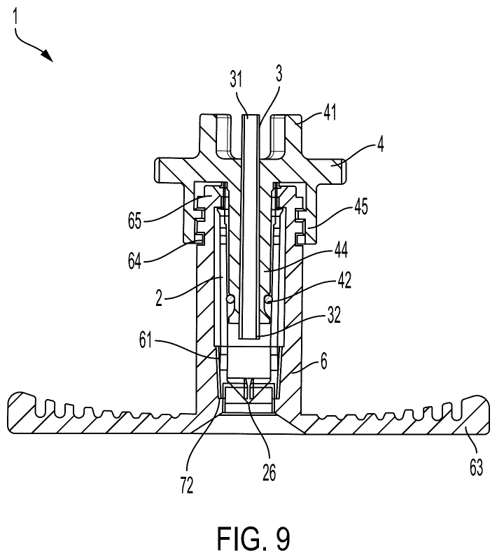

The sample holder in FIG. 8 is different from that in FIG. 7 in that the cap 4 is configured to form a seal with an inner surface of the vessel 2 wall, rather than an outer surface as in FIGS. 1-8. FIG. 9 shows a cross sectional view of a sample holder like that in FIG. 8 and which include a cap arranged to form a seal with an inner surface of a vessel wall. For example, in some cases a cap 4 can have a wall 44 that is configured to be inserted into the internal space of the vessel 2. In some cases, the wall 44 can have a cylindrical or other tubular shape that is sized and shaped to fit within the internal space of the vessel 2. The wall 44, e.g., at a lower end, can carry a seal 42 configured to form an airtight seal with an inner surface of the vessel 2 wall that defines the internal space. Thus, as the cap 4 is moved to withdraw the wall 44 from the internal space of the vessel 2, a pressure gradient may be generated between the proximal and distal ends of the conduit 3 that causes a liquid sample and/or gas in the conduit 3 to move into from the proximal end 31 toward the distal end 32. To provide a vent so a sample can be received into the conduit 3, the inner surface of the vessel 2 can include one or more channels (such as the channels 21 in FIG. 1) or otherwise be configured so that an airtight seal is not created between the cap 4 and the vessel 2. In some cases, the vessel 2 may have a diameter or other size or shape configured so that the seal 42 does not contact the vessel wall to define an airtight seal. In some embodiments, the cap 4 may be configured with one or more channels or other features to prevent the creation of an airtight seal. For example, a groove in the wall 44 where the seal 42 is carried by the cap 4 may be sized and/or shaped so that when the cap 4 is inserted into the vessel 2, the seal 42 moves into a portion of the groove (e.g., at an upper section of the groove) that is configured to prevent formation of an air tight seal (e.g., because of the presence of channels, a reduced diameter or other size where the seal 4 contact the wall 44, etc.). However, when the cap 4 is moved to remove the wall 44 from the vessel 2, the seal 42 may engage a different portion of the groove (e.g., a lower section of the groove) that permits an airtight seal to be created. Other arrangements are possible, including one way valves of the cap 4 and/or vessel 2.

Movement of the cap 4 relative to the vessel 2 may be like that in FIG. 1, e.g., a user may be freely permitted to move the cap 4 relative to the vessel 2, whether in a linear direction along a longitudinal axis of the conduit 3 and/or vessel 2 or rotationally about the longitudinal axis. However, in some cases, the cap 4 may be configured to engage with the vessel 2, a stand 6 or other component so that movement of the cap 4 relative to the vessel 2 is restricted or otherwise controlled in some way. For example, in some cases, the cap 4 may be threadedly engaged with the vessel 2 and/or stand 6 so that rotation of the cap 4 relative to the vessel 2 and/or stand 6 moves the cap 4 in a linear direction, e.g., along a longitudinal axis of the vessel 2 or otherwise to remove the cap 4 from the vessel 2. One such arrangement is shown in FIG. 9 where the cap 4 is threadedly engaged with a stand 6. Rotation of the cap 4 relative to the stand 6 moves the cap 4 in an up and down direction, e.g., so the wall 44 of the cap 4 is moved in a direction to move into and out of the vessel 2. Thus, for example, the cap 4 may be rotated counterclockwise relative to the stand 6 to remove the cap 4 and its wall 44 from the vessel 2 (to move a sample in the conduit 3 into the vessel 2) and may be rotated clockwise to engage the cap 4 with the vessel 2 and move the wall 44 into the internal space of the vessel 2 (to ready the sample holder 1 to receive a sample). In some embodiments, a threaded engagement between the cap 4 and stand 6 or vessel 2 may help control movement of the cap 4 relative to the vessel 2. For example, it may be desirable to move a liquid sample from the conduit 3 into the vessel 2 at a slow or otherwise controlled rate, e.g., to prevent splashing or splattering of the sample in the vessel. A threaded engagement can help control movement of the cap 4 relative to the vessel 2, e.g., to prevent rapid movement of the cap 4 relative to the vessel 2. A threaded engagement can in some cases help maintain the cap 4 in a suitable orientation relative to the vessel 2, e.g., in a vertical orientation, which may help ensure a suitable seal is created and maintained between the cap 4 and vessel 2. In some cases, the cap 4 can have an internal thread 45 configured to engage with an external thread 64 on the stand 6 to move the cap 4 relative to the stand 6 and a vessel 2 held by the stand 6, e.g., in a cavity 61 of the stand 6. In some cases, the stand 6 may include a collar 65 configured to engage with a portion of the vessel 2 to help hold the vessel 2 in the cavity 61 of the stand 6. For example, the collar 65 may include an annular ring that extends radially inwardly into the cavity 61 and is configured to engage with a rim or flange of the vessel 2, e.g., to help prevent movement of the vessel 2 out of the cavity, such as along its longitudinal axis in an upward direction. Thus, after a sample has been received in the conduit 3, the cap 4 can be unthreaded from the stand 6 to move the cap 4 upwardly relative to the stand 6 and vessel 2, e.g., so the cap 4 can be removed from the vessel 2 while the vessel 2 remains held in the cavity 61. Thereafter, a cover 5 can be engaged with the vessel 2, e.g., to seal the opening to the internal space of the vessel 2 closed. In some cases, the stand 6 and vessel 2 may have complementary engagement features to help prevent movement of the vessel 2 relative to the stand 6, e.g., in a rotational direction. For example, the stand 6 may include engagement features 72 that engage with corresponding features 26 on the vessel 2. For example, the engagement features 72 may include recesses, fins, tabs, etc. that engage with a corresponding recess, fin, tab, etc. on the vessel 2 to help prevent rotation of the vessel 2 relative to the stand 6.

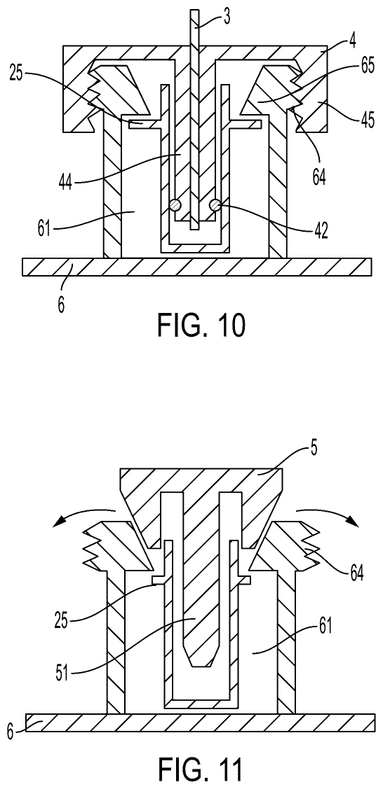

FIG. 10 shows a schematic diagram of a sample holder 1 arrangement like that in FIG. 9. As can be seen in FIG. 10, the collar 65 may have a portion that engages with a flange 25 or other portion of the vessel 2, e.g., such as an upper rim at the opening to the internal space, to help keep the vessel 2 in the cavity 61. Thus, the cap 4 can be unthreaded from its engagement with the thread 64 on the stand 6. It should be noted that in some embodiments the cap 4 may threadedly engage with the vessel 2, e.g., threads 22 on the vessel 2, rather than or in addition to a threaded engagement with a stand 6. With the cap 4 removed from the vessel 2, a cover 5 can be engaged with the vessel 2, e.g., as shown in FIG. 11. In some embodiments, engagement of a cover 5 with the vessel 2 can serve to disengage the vessel 2 from the stand 6 or otherwise permit removal of the vessel 2 from the stand 6. For example, in some embodiments, the cover 5 may have one or more portions that engage with the collar 65 or other portion of the stand 6 to free a flange 25 or other portion of the vessel 2 from engagement or other interference by the collar 65. In some cases, the cover 5 may have a tapered or otherwise configured portion that engages with the collar 65 to bias portions of the collar 65 away from each other, e.g., to move portions of a rim or other part of the stand 6 away from a flange 25 or other part of the vessel 2 so the vessel 2 can be removed from the cavity 61. The collar 65 can include one or more slots, e.g., similar to the window 66 in FIG. 6, to help permit portions of the collar 65 to be moved away from each other. In addition, or alternately, the portion of the stand 6 that engages with the vessel 2 could be made of a resilient or otherwise flexible material. The cover 5 can engage with the vessel 2 in any suitable way, such as by friction fit, threaded engagement with threads 22 on the vessel 2, etc. For example, threading of the cover 5 onto the vessel 2 could cause the collar 65 to be moved to free the vessel 2 from the stand 6.

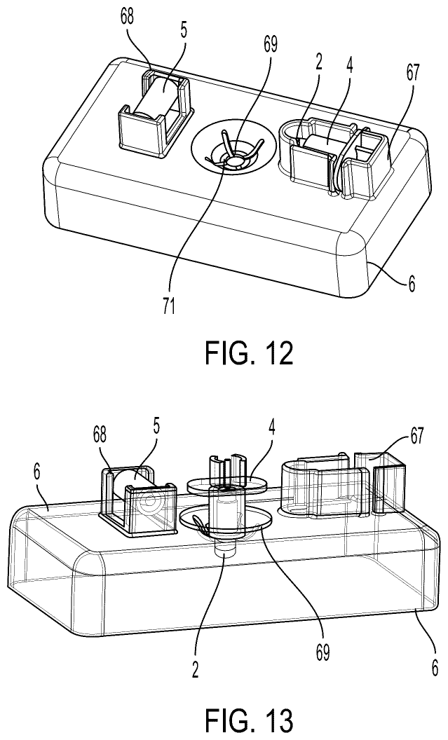

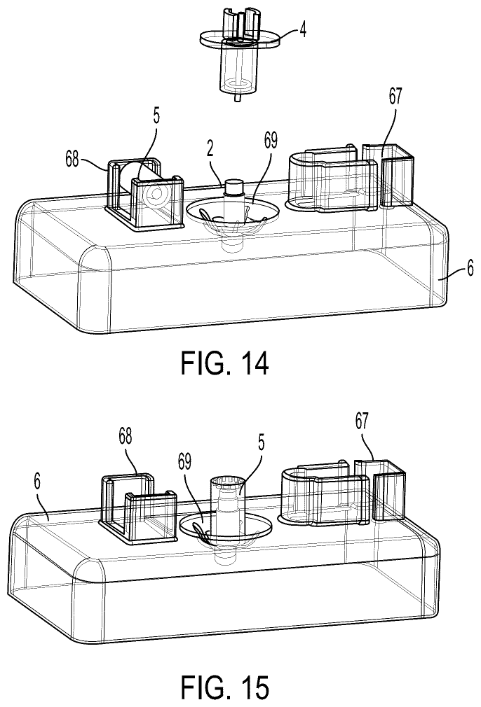

In some embodiments, a stand or other holder for one or more components of a sample holder can be configured to assist for multiple functions, such as shipping in a small form factor, presenting components of the sample holder in a convenient way for use, and/or others. FIG. 12 shows a perspective view of a stand 6 that is configured to hold an assembled vessel 2 and cap 4 in a first location 67, and hold a cover 5 in a second location 68. The stand 6 may be configured to be supported on a surface, such as a tabletop, so that a user can easily access both locations 67, 68. A third location 69 may be provided to receive a vessel 2, e.g., with an engaged cap 4, but may be initially provided with no component held at the third location 69. As an example, the configuration in FIG. 12 may be packaged, e.g., in a bag or box, that can help maintain the sample holder 1 in a sterile or otherwise suitably clean environment until use and can be opened by a user to access the stand 6. A user may remove the assembled cap 4 and vessel 2 from the first location 67 and place the vessel 2 at the third location 69, e.g., so the vessel 2 and cap 4 are in an upright position as shown in FIG. 13. In some cases, the third location 69 may include engagement features such as spring fingers 71 configured to engage with a portion of the vessel 2, such as at the recess 23 in an embodiment like that in FIG. 1. The spring fingers 71 may resiliently engage the vessel 2 so the vessel 2 and cap 4 are held upright without assistance from a user. In addition, the spring fingers 71 may resist movement of the vessel 2 in a vertical direction, e.g., along a longitudinal axis of the vessel and/or the conduit 3. With the cap 4 and vessel 2 held at the third location 69, a blood or other sample may be provided to the conduit 3, e.g., by capillary action. Thereafter, the cap 4 may be removed from the vessel 2 as shown in FIG. 14, e.g., by pulling upwardly on the cap 4. The vessel 2 may be held at the third location 69 by the spring fingers 71 or other engagement features so that a user need not grasp the vessel 2 individually and instead may hold only the stand 6. The removed cap 4 may be discarded or placed in the first location 67, and the cover 5 removed from the second location 68 and engaged with the vessel 2 at the third location 69, e.g., as shown in FIG. 15. The assembled vessel 2 and cover 5 can be packaged and shipped, as needed, in the arrangement shown in FIG. 15, or the vessel 2 and cover 5 can be removed from the stand 6 and processed or otherwise handled in any suitable way.

Note that a user of a sample holder can employ the sample holder for one or more different functions. For example, in some cases a user may employ a sample holder only for sample collection. Such a user may collect a sample from the user, e.g., a person may express a blood droplet and use the sample holder to collect the sample, or a person may use a sample holder to collect a sample from another person or other subject. This user may close the sample holder after collection, e.g., by placing a cap on a vessel, and have no further interaction with the sample holder. In some cases, a user may employ a sample holder for treatment and/or analysis of a sample that was previously collected, e.g., by another person. Thus, a user for treatment and/or analysis need not use a sample holder for sample collection and may interact with the sample holder in other ways, such as by opening the vessel. Reagents or other materials may be added to the vessel 2, e.g., for processing of the sample in the vessel 2, and/or sample material in the vessel 2 may be removed for treatment and/or analysis, and/or a sample in a vessel may be treated without opening of the vessel 2. Any such users may employ one or more features of a sample holder and/or stand, or not.

The vessel 2 can be made of any suitable material or combination of materials. Different materials may be employed to help the different vessel portions perform their functions. For example, the vessel may be made of a material that is suitable for exposing a sample in the holder 1 to focused acoustic energy, heat or other treatment conditions, e.g., for shearing blood cells and/or nucleic acids, PCR amplification, etc. Portions of the vessel 2 can be made of a material that provides a suitable sealing surface with the cap 4 without providing excessive friction that resists movement of the cap 4 relative to the vessel 2. Example materials for the vessel components include polyethylene, polypropylene, glass, metal, etc.

While aspects of the invention have been described with reference to various illustrative embodiments, such aspects are not limited to the embodiments described. Thus, it is evident that many alternatives, modifications, and variations of the embodiments described will be apparent to those skilled in the art. Accordingly, embodiments as set forth herein are intended to be illustrative, not limiting. Various changes may be made without departing from the spirit of aspects of the invention.

1. A sample holder comprising:
a vessel having a wall defining an internal space and an opening to the internal space;
a conduit having a distal end positioned in the internal space and a proximal end extending from the internal space, the conduit configured to receive a liquid sample into the proximal end of the conduit; and
a cap configured engage with the vessel such that movement of the cap relative to the vessel from a first position toward a second position causes a pressure differential between the proximal and distal ends of the conduit suitable to move a liquid sample from the conduit into the internal space.

2. The sample holder of claim 1, wherein the cap is configured to cause a pressure below ambient pressure in the internal space in response to movement of the cap relative to the vessel.

3. The sample holder of claim 2, wherein the pressure below ambient pressure is suitable move a liquid sample from the conduit into the internal space via the distal end.

4. The sample holder of claim 1, wherein the cap is configured to cause the pressure differential in response to rotation of the cap relative to the vessel.

5. The sample holder of claim 1, wherein the cap is configured to cause the pressure differential in response to linear movement of the cap relative to the vessel.

6. The sample holder of claim 1, wherein the cap is configured to sealingly engage with an outer surface of the wall of the vessel.

7. The sample holder of claim 6, wherein a portion of the outer surface of the wall includes a channel that provides a vent for the internal space when the cap is at the first position.

8. The sample holder of claim 1, wherein the cap is configured to sealingly engage with an inner surface of the wall of the vessel.

9. The sample holder of claim 1, a portion of the inner surface of the wall includes a channel that provides a vent for the internal space when the cap is at the first position.

10. The sample holder of claim 1, wherein the conduit includes a capillary tube configured to draw a blood sample into the capillary tube by contacting blood to the proximal end.

11. The sample holder of claim 1, wherein the conduit is fixed to the cap.

12. The sample holder of claim 11, wherein the cap and the conduit are removable from the vessel by moving the cap from the first position to the second position.

13. The sample holder of claim 12, further comprising a cover configured to engage with the vessel to close the opening and seal the internal space closed with the cap and conduit removed from the vessel.

14. The sample holder of claim 13, further comprising a desiccant associated with the cover, positionable in the internal space and configured to dry a blood sample enclosed in the internal space.

15. The sample holder of claim 14, wherein the desiccant extends from the cover and is configured to be positioned in the internal space with the cover engaged with the vessel.

16. The sample holder of claim 1, wherein the conduit extends from an upper side of the cap and the cap includes a guard that extends from the upper side of the cap adjacent the proximal end of the conduit.

17. The sample holder of claim 1, further comprising a holder configured to engage with the vessel and hold the vessel in an upright position on a surface with the proximal end of the conduit extending upwardly from the vessel.

18. The sample holder of claim 17, wherein the holder and the vessel include complementary engagement features to removably engage the vessel with the holder.

19. The sample holder of claim 17, wherein the cap and the holder are configured to support the vessel and conduit in a horizontal position on the surface with the conduit extending horizontally.

20. A method of collecting a blood sample, comprising:
providing a cap engaged with a vessel and conduit having a distal end within an internal space of the vessel, the vessel including a wall that defines the internal space;
contacting blood with a proximal end of a conduit to draw the blood into the conduit; and
removing the cap from the vessel to create a pressure differential between the proximal end and the distal end of the conduit suitable to move the blood out of the conduit and into the internal space of the vessel.

21. The method of claim 20, wherein removing the cap includes moving the cap relative to the vessel so that the cap engages with different portions of the vessel.

22. The method of claim 21, wherein removing the cap includes engaging the cap with different portions of the vessel wall.

23. The method of claim 22, wherein removing the cap includes engaging the cap with different portions of an inner surface of the vessel wall.

24. The method of claim 22, wherein removing the cap includes engaging the cap with different portions of an outer surface of the vessel wall.

25. The method of claim 20, wherein removing the cap includes moving a gasket of the cap along a surface of the vessel wall.

26. The method of claim 20, wherein contacting blood with the proximal end includes venting gas from the internal space as blood is drawn into the conduit.

27. The method of claim 26, wherein venting gas includes venting gas through a space between the cap and the vessel wall.

28. The method of claim 20, wherein removing the cap includes removing the conduit from the vessel with the cap.

29. The method of claim 20, wherein removing the cap includes rotating the cap relative to the vessel.

30. The method of claim 20, further comprising engaging a cover with the vessel to seal the internal space closed after the cap is removed from the vessel.

31. The method of claim 20, wherein engaging the cover includes positioning a desiccant attached to the cover in the internal space, the desiccant being suitable to dry the blood sample.

32. The method of claim 20, wherein providing the cap includes providing the conduit fixed to the cap.
